(19) United States
(12) Patent Application Publication
(10) Pub. No.: US 2013/0133667 A1
(43) Pub. Date: May 30, 2013

(54) INTRA-UTERINE SYSTEM

(71) Applicants: Antonius Jozef Fredericus Heijm, Nijkerk (NL); Francine van Os-Bardiaux, Monte Carlo (MC)

(72) Inventors: Willem Arthur Adriaan van Os, Monte Carlo (MC); Antonius Jozef Fredericus Heijm, Nijkerk (NL)

(73) Assignees: Willem Arthur Adriaan Van Os, Monte Caarlo (MC); HEIJMCOMB B.V., Nijkerk (NL)

(21) Appl. No.: 13/509,066

(22) PCT Filed: Nov. 11, 2010
(86) PCT No.: PCT/NL10/50749

§ 371 Date: Jul. 18, 2012

(30) Foreign Application Priority Data
Nov. 12, 2009 (NL) 2003794

(51) Int. Cl. A61F 6/14

(52) U.S. Cl. CPC A61F 6/14
USPC 128/833

(57) ABSTRACT

Intra-uterine system for inserting an active substance in an uterus, wherein the system comprises two flexible arms that extend from a central part in essentially mutually opposed directions, and retention means for the active substance, wherein during use the system is entered into the uterus, wherein an elongated housing extends from the central part at essentially equal angles with the flexible arms, wherein the housing comprises a storage container as retention means for the active substance, an outlet opening, a pump system, a control unit for controlled pumping of the active substance from the storage container through the outlet opening into the uterus and a energy storage means for supplying energy to the pump system and to the control unit.

The invention relates to an intra-uterine system for inserting an active substance in a uterus, wherein the system comprises two flexible arms that extend in generally opposite directions from a central part, and retaining means for the active substance, wherein during use the system is inserted in the uterus.

Such a system is known, particularly as a contraceptive wherein the active substance is a copper spiral that is attached to the central point. After inserting the system into the uterus the copper spiral will emit copper ions. Copper ions are known to have a good contraceptive effect. Which such a system it is further important that the system is conceived such that the external dimensions are small, because if not the presence of the system in the uterus can have a disturbing effect and the system must also be arranged such that the possibility for damages to the uterus are minimal. Although the use of a copper spiral as a contraceptive means has enabled the manufacture of such a system with small external dimensions, the possibility remains that an intra-uterine system that is made of a material such as copper, which in addition has the form of copper wire wound into a spiral, is hazardous with respect to damages to the uterus. In addition the process of dissolving of the copper in the fluids that are present in the uterus and hence emission of copper ions, is a process that is uncontrolled and can vary from case to case with respect to the amount and concentration in which the copper ions are emitted. This gives an amount of uncertainty with respect to the effectiveness of these known systems.

In addition to systems with a copper spiral, systems that instead of a copper spiral comprise a tubular holder that contains a hormone, e.g. a progestativum are also known (such as described in EP 0 673 629 A1). Also here the emission of the active substance takes place in a non-controlled way.

From DE 101 45 269 A an intra-uterine device is known comprising a support element with pharmaca depots attached to it that can be emptied in a defined way by control elements. The pharmaca depots that protrude from the support element give an increased risk for damage to the uterus. Inserting the intra-uterine device of DE 101 45 269 A takes place e.g. with an applicator as has been described in DE 198 15 552. This is an applicator wherein the intra-uterine device is places in a sleeve that is being entered into the uterus. The intra-uterine device is subsequently pushed out of the sleeve by means of a pushing stamp. This way of inserting is cumbersome and may easily lead to damaging the uterus. In addition, with the applicator as has been described in DE 198 15 552, a proper positioning of the flexible arms abutting the fundus is not possible or at least not easily possible. The protruding pharmaca depots that have been mentioned above prevent the use of an applicator as has been described in WO 2007/075086, which enables an effective and simple way of inserting and positioning of an intra-uterine device.

It is the aim of this invention to remedy the disadvantages mentioned, respectively to further improve the existing devices.

This aim has been reached by an intra-uterine system according to claim 1. With such a system the housing that accommodates a container for the active substance can be executed in such a way that the possibilities for damaging the uterus are very small, for instance by a choice of materials and a suitable finishing of the external surfaces of the housing. In addition, with the design described an appluicator can be used as has been described in WO 2007/075086, so that an effective and simple method of inserting and positioning of an intra-uterine device in the uterus is possible. Furthermore the active substance is brought into the uterus in an active way by the control and the pump system, so that the amount of active substance that is brought into the uterus by the system according to the invention can be controlled. In addition developments in e.g. micro- and nanotechnologies enable the design of pump systems of very small dimensions. By using a controlled pump system for issuing the active substance, the amount of active substance issued can be metered very accurately which enables the system to issue a controlled amount of active substance.

In a simple and conveniently working embodiment of the invention, the system comprises a switching means for switching the system on and off. This enables the system to be switched on e.g. directly before bringing the system into the uterus. The switching means can be executed in a simple way as connecting respectively disconnecting the supply of energy of the energy storage means to the energy users of the system. To a person skilled in the art many possibilities for this are available.

In a preferred embodiment the control unit comprises a microprocessor with a time clock and a memory. A thus equipped control unit not only has the advantage that it can be manufactured with very small dimensions, but also opens the possibilities to execute the control by means of a program stored in the memory, wherein the program uses parameters that are also stored in memory, which make the control unit easily adaptable to varying conditions, which makes the control unit very flexible.

In an advantage embodiment of the invention the control unit is arranged to pump a predetermined amount of active substance into the uterus at predetermined time intervals. This predetermined amount may be constant during the predetermined intervals or may vary with successive intervals. It is also possible to either keep the intervals constant or vary the intervals. It has to be understood as follows. During a certain time interval a predetermined amount of active substance is pumped in the uterus. It may be that the amount of active substance that is pumped into the uterus per time unit is constant during the whole interval. However, it is also possible that during a part of the interval active substance is being pumped into the uterus and during the remaining part of the interval no active substance is being pumped into the uterus.

I a preferred embodiment the invention comprises a control unit that is equipped with a transceiver, wherein the microprocessor is coupled to the transceiver and to the switching means. This enables remote communication with a system according to the invention after it has been entered into a uterus. This makes it possible for instance to switch on a system that has been entered into the uterus and also to switch it off again. However, also the programming of the control unit of the system, as has been described above, can be modified after the system has been entered into the uterus. This obviously requires an external communication unit. Such communication units are available in the market and do not have to be discussed here in more detail. However, it has to be ensured that the system is arranged in such a way that the external communication is protected against unauthorized use. However, also in this case it will be clear for a person skilled in the art how to this needs to be arranged and therefore it does not need to be discussed here in more detail.

In a preferred embodiment of the invention the active substance is being stored in the storage container in liquid form or in a form dissolved in a liquid. Applying a pump system to such an essentially liquid form is extremely simple. However, the invention is not limited to this because also other pumpable media such as in a form of a gas or in a form of fine grains can be issued into the uterus with a pump system.

When the energy storage means comprises a dry cell battery this not only enables the energy storage means to have small dimensions, but also has the advantage that electric energy has a wide applicability. However, in spite of the clear advantages of a dry cell battery the invention need not to be limited to this and also for instance pneumatic energy storage means is possible.

In a preferred embodiment the length of the housing lies between 21 tot 26 mm and preferably amounts to 21 mm and the housing has a maximum diameter of 7 mm, preferably 5-6 mm. Within these dimensions it is possible with the presently known technologies, for instance with nanotechnology, to accommodate all necessary parts and also a proper amount of the active substance. Advantageous embodiments comprise a pump system with a volumetric membrane pump integrated on a MEMS chip (Micro Mechanical Electrical System) or a pump system based on electro osmotic flows (EO).

In a further preferred embodiment of the invention the storage container has a variable volume. For instance this can be obtained by the storage container being formed by a pouch made from a thin foil that is accommodated in the housing. In such a case, when there is active substance pumped out of the storage container, the pouch will simply decrease in volume by the ambient pressure and thus there will be no vacuum inside the storage container but always the ambient pressure will be maintained, and thus there are no additional measures necessary to avoid vacuum in the storage container.

A very stable position of the intra-uterine system according to the invention appears to be obtained when the free ends of the flexible arm are spherical and in use are resting against the fundus, each in a utero tubal corner. Because the uterus is continuously in motion and contractions can expel an intra-uterine system, it is important that the stable position of the system according to the invention is reached. This is notably the case because the spherical ends of the flexible arm provide for a fundus seeking effect, such that notably during contractions of the uterus the two arms with the spherical ends maximally prevents expulsion of the intra-uterine system.

This effect is being amplified when the flexible arms have such a curvature that during use the arms remain essentially free of the fundus. Through this the contact with the fundus will mainly take place via the spherical ends of the flexible arms, which has a stabilizing effect. Because the ends have a spherical embodiment these spherical contacts with the fundus of the uterus will not damage the uterus.

The non-damaging properties of the intra uterine system according to the invention is being amplified when the housing is flexible and consequently can somewhat accommodate the movements that are always present in the uterus. When the housing has essentially smooth external contours, even when there is contact with the uterus wall or with the fundus, this contact will not or hardly result in damages. Further characteristics and advantages of the invention will be explained in the description of an example of an embodiment of the invention, also with reference to the drawing in which:

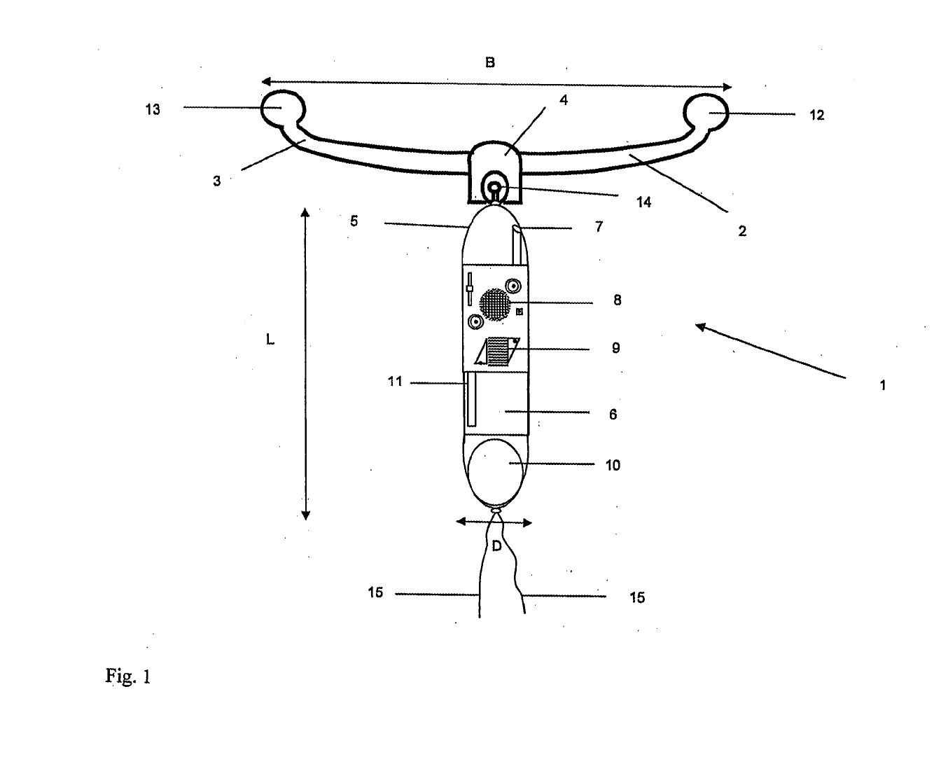

FIG. 1 shows a schematic reproduction in cross section of a first embodiment of a intra-uterine system according to the invention;

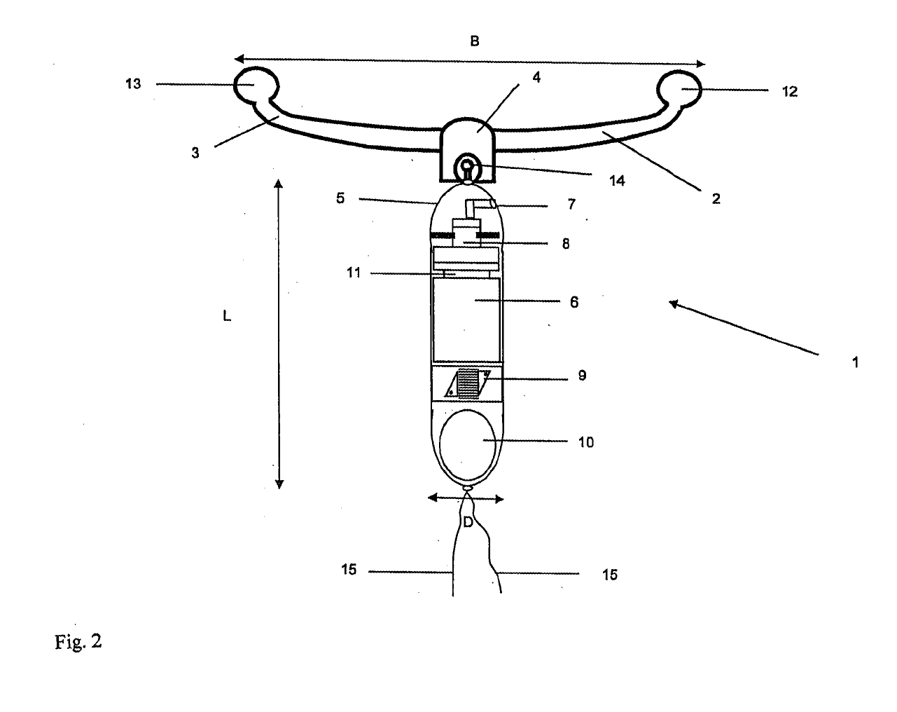

FIG. 2 shows a schematic reproduction in cross section of a second embodiment of an intra-uterine system according to the invention.

In each of the FIGS. 1 and 2 an embodiment of the intra-uterine system according to the invention is referenced as a whole with number 1. From a central part 4 two flexible arms 2, 3 extend in mutually opposite directions. An elongated housing 5 also extends from central part 4. Housing 5 is connected with central part 4 by a pivoting attachment 14. Housing 5 comprises a reservoir 6 that is filled with an active substance, a pump system 8 with a control unit 9, which in this case are integrated to one unit, which will be discussed later in more detail, and energy storage means 10 in a form of a dry cell battery. The dry cell battery 10 is electrically connected with a pump system 8 and with control unit 9. Further an outlet opening 7 is shown. Outlet opening 7 discharges outside the housing 5, during use into the uterus.

With the embodiment of the intra-uterine system 1 according to the invention that is shown in FIG. 1, further an inlet 11 can be seen. Inlet 11 of the pump systems extends into container 6 for the active substance. When the pump system is in operation, active substance is being pumped from reservoir 6 via inlet 11 through the pump system 8 and via outlet opening 7 to the external surroundings of the intra-uterine system 1. During use the intra-uterine system 1 is placed in the uterus and thus the active substance that is pumped through the pump system and through the outlet opening 7 to the exterior, is being pumped into the uterus.

In the embodiment of the system 1 of the invention that is shown in FIG. 1, the pump system 8 and the control unit 9 are integrated in a so called MEMS (Micro Electro Mechanical Systems) chip. This chip comprises micromechanically manufactured pump structures that operate according to the principle of a volumetric membrane pump, a microprocessor with electronic time switch structures and also a piezo-electric actuator that gives an alternating movement to the pumping membrane. Valves have been fitted in the inlet 11 and in the outlet 7 of the pump system 8 so that the membrane pump can pump the active substance from storage container 6 to the uterus outside the outlet opening 7.

FIG. 2 shows an embodiment of the invention with a pump system 8 that is based on electro-osmoses (EO). Such a pump system comprises a porous wall of a suitable material that separates in this case storage container 6 and outlet opening 7. The porous wall comprises capillary micro channels that connect storage container 6 with outlet opening 7. By applying a suitable electrical field over the porous wall, fluid is moved through the capillary channels and thus a pumping action is created. This has the advantage over other types of micro pumps that it does not have any moving parts, the operation is very simple and is based on direct electrical control. The EO pump 8 is connected with control unit 9. It is also possible to integrate control unit 9 and pump system 8 in a micro fluidic chip, that comprises micro channels for pumping of a fluid with the active substance, and also an electronic timing circuit, memory and a central processing unit to bring a predetermined amount of active substance into the uterus within a predetermined period or with a predetermined interval.

In the examples shown of the intra-uterine system 1 according to the invention, the microprocessor comprises a memory in which both a control program as well as operating parameters is stored for the pumping process to be used. In such a way a predetermined amount, controlled by the stored parameters, of active substance can be pumped into the uterus. The pump system then stops for a period of time after which again a determined amount of the active substance is being pumped from the storage container 6 to the uterus. By controlling the time during which the pumping takes place or by controlling the amount of pump strokes, the amount of active substance to be pumped can be controlled. Under control of the program that amount can be constant each time, but it is also possible to vary this amount each time. It is also possible to vary the intervals between pumping out the predetermined amount of active substance. All this can be depending on the application for which the intra-uterine system is being used. For instance for use of the intra-uterine system 1 according to the invention as a contraceptive means, the amount of pumped-out substance can be constant each time and the intervals are likely to be constant. For instance when the active substance is a solution of CuCl₂, each 24 hours so much of this solution can be pumped into the uterus that by this 80 μg/day of copper ions are being pumped into the uterus.

Through this a good and reliable contraceptive action of the system 1 is being insured. When the intra-uterine system 1 according to the invention is being used for applying medication on a therapeutical basis, the amount and frequency can be adapted to the prescriptions of the MD responsible. To this end, prior to inserting the intra-uterine system 1 into the uterus, the desired program with the desired parameters are loaded into the microprocessor, so that medication can take place following the prescriptions of the MD responsible.

In both above-mentioned examples of embodiments of the uterine system 1 according to the invention the control unit 9 comprises a transceiver that is coupled to the microprocessor. This enables communication with a communication unit for example that is not shown here. For instance after inserting the system into the uterus, with the external communication unit the system can be switched on for instance by starting the above-mentioned program. Also this program can be stopped and can be modified if necessary. It will be clear that this communication needs to be secured against unauthorized use. Also the reach of the transceiver of control unit 9 as well as from the communication unit will be limited to 1-2 m. This allows for directional communication with an intended specimen of a system 1 according to the invention, and it can be avoided that unintended more than one system is being activated.

The flexible arms 2, 3, the central part 4 and the housing 5 are made of a plastic that is compatible with the uterus. The application of the so called nanotechnologies to the manufacture of the pump system 8 and the control unit 9, the dimensions of the housing 5 can be kept sufficiently small, so that a safe intra-uterine system 1 can be obtained that is well tolerated by the uterus. The length L of the shown embodiment of the housing will be between 21 and 26 mm and will amount 21 mm preferably, and the largest diameter D of this housing will be between 5 and 6 mm. Because all parts have a smooth external shape, the possibility of damaging the uterus will be minimal.

Further it is noted that the flexible arms 2, 3 have a slightly arched form and end in, in the figure shown upwards oriented spherical shaped ends 12, 13. When positioning the intra uterine system 1 according to the invention the system will be placed in such a way that the spherical shaped ends 12, 13 rest against the fundus and each of the arms will be situated in one of the two utero-tubal corners. The nominal width B, being the distance between both ends, amounts to 32 mm. The slight arcuation of the both arms 2, 3 is chosen such that when both spherical shaped ends 12, 13 rest against the fundus of the uterus, both arms are essentially situated in a small distance from the fundus, but follow the contour of the fundus. Because of this a very stable position of the intra-uterine system 1 is obtained, notably because the support takes place at both ends of the arms, using the available symmetry in the uterus. Because the ends 12, 13 have been chosen as spherical shaped, the possibility for damaging the fundus is very small, which is notably important for the occurring contractions of the uterus.

The embodiments of the invention in FIGS. 1 and 2 further show that to the housing 5, near the end that is away from the central part 4 flexible threads 15 are connected. The threads 15 serve to remove the intra-uterine system 1 from the uterus after use.

With the intra-uterine system 1 according to the invention a system is obtained with which in a reliable way a controlled dose of a active substance can be administered into the uterus with the action of inserting having to take place only once, and after ending the medication or after exhausting the reservoir the one time action of removing again the system 1 from the uterus is necessary. Between these two moments the medication takes place completely automatically and according the schedule of the responsible doctor, which is being reflected in process parameters that have been programmed into the memory of the control unit 9. Also the fact that the medication is administered directly there where its effect is required, gives the advantage that no further system of the body of the patient needs to be burdened with the active substance.

Although two examples of embodiments of an intra-uterine system 1 according to the invention have been mentioned, it will be clear that many variations are possible that all are covered by the scope of the invention as described in the attached claims.

### List of Reference Numbers

1 Intra-uterine system
2 Flexible arm
3 Flexible arm
4 Central part
5 Housing
6 Storage container
7 Outlet opening
8 Pump system
9 Control unit
10 Energy storage means
11 Inlet of pump system
12 Spherical end of 2
13 Spherical end of 3
14 Pivoting attachment
15 Flexible threads

1. Intra-uterine system for inserting an active substance in a uterus, wherein during use the system is entered into the uterus, wherein the system comprises two flexible arms that extend from a central part in generally mutually opposed directions, and retention means for the active substance, wherein an elongated housing extends from the central part at essentially equal angles with the flexible arms, wherein the housing comprises
a storage container as retention means for the active substance,
an outlet opening,
a pump system,
a control unit for controlled pumping of the active substance from the storage container through the outlet opening into the uterus and an
energy storage means for supplying energy to the pump system and to the control unit, wherein
the housing is formed by a cylinder with a flush external surface that
at both ends is joined to a closing surface
comprising a flowing external contour and
a flowing transition to the external surface of the cylinder,
the housing comprising a diameter of less than or equal to 6 mm and a length of less than or equal to 26 mm.

2. Intra-uterine system according to claim 1, wherein the system comprises a switching means for switching the system on and switching the system off.

3. Intra-uterine system (1) according to claim 1, wherein the control unit comprises a micro processor with a time clock and a memory.

4. Intra-uterine system according to claim 1, wherein the control unit is arranged to pump a predetermined amount of active substance at predetermined time intervals from the storage container into the uterus.

5. Intra-uterine system according to claim 4, wherein the amount of active substance is variable per time interval.

6. Intra-uterine system according to claim 4, wherein the length of the time intervals are variable.

7. Intra-uterine system according to claim 3, wherein the control unit comprises a transceiver, wherein the microprocessor is coupled to the transceiver and to the switching means.

8. Intra-uterine system according to claim 1, wherein the active substance in the storage container is stored in liquid form or dissolved in liquid.

9. Intra-uterine system according to claim 1, wherein the energy storage means comprises a dry cell battery.

10. Intra-uterine system according to claim 1, wherein the housing has a length (L) of 21 to 26 mm.

11. Intra-uterine system according to claim 1, wherein the housing has a maximum diameter (D) of 5 to 6 mm.

12. Intra-uterine system according to claim 1, wherein the pump system comprises a volumetric membrane pump integrated in a MEMS (Micro Mechanical Electrical System).

13. Intra-uterine system according to claim 1, wherein the pump system comprises a pump system based on electro osmosis (EO).

14. Intra-uterine system according to claim 1, wherein the storage container has a variable volume.

15. Intra-uterine system according to claim 1, wherein the free ends of the flexible arms are of spherical shape and in use are resting against the fundus, each in a utero-tubal corner.

16. Intra-uterine system according to claim 15, wherein the flexible arms are arcuated in such a way that during use the arms remain essentially free from the fundus.

17. Intra-uterine system according to claim 1, wherein the housing is flexible.
